(19) United States
(12) Patent Application Publication
(10) Pub. No.: US 2011/0112104 A1
(43) Pub. Date: May 12, 2011

(54) TRICYCLIC COMPOUNDS, A PROCESS FOR THEIR PREPARATION AND PHARMACEUTICAL COMPOSITIONS CONTAINING THEM.

(75) Inventors: Patrick Casara, Villennes Sur Seine (FR); Thierry Le Diguarher, Rueil Malmaison (FR); Didier Durand, Chambourcy (FR); Olivier Geneste, Rueil-Malmaison (FR); John Hickman, Paris (FR)

(73) Assignee: LES LABORATOIRES SERVIER, Suresnes Cedex (FR)

(21) Appl. No.: 12/737,436

(22) PCT Filed: Jul. 10, 2009
(86) PCT No.: PCT/FR2009/000855

§ 371 Date: Jan. 13, 2011

(30) Foreign Application Priority Data
Jul. 15, 2008 (FR) 08/04014

(51) Int. Cl. A61K 31/4985; C07D 471/14; C07D 241/36; A61K 31/498; A61P 35/00

(52) U.S. Cl. 514/250; 544/346; 544/344

(57) ABSTRACT

Compounds of formula (I):

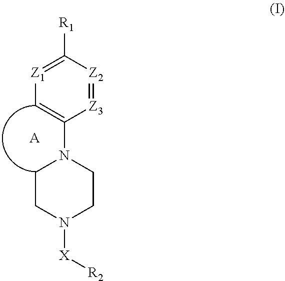

wherein:
A represents a 5, 6 or 7-membered (hetero)aromatic or non-aromatic ring,
Z₁, Z₂ and Z₃ each independently of the others represents a CH group or a nitrogen atom, it being understood that at least one of these three groups is a nitrogen atom,
X represents an alkylene chain as defined in the description,
R₂ represents an aryl or heteroaryl group,
the group R₁ represents a group of formula (II) as defined in the description

Medicinal products containing the same which are useful in treating conditions involving a defect in apoptosis.

The present invention relates to new tricyclic compounds, to a process for their preparation and to pharmaceutical compositions containing them.

The compounds of the present invention are new and have very valuable pharmacological characteristics in the field of apoptosis and cancerology.

Apoptosis, or programmed cell death, is a physiological process that is crucial for embryonic development and maintenance of tissue homeostasis.

Apoptotic-type cell death involves morphological changes such as condensation of the nucleus, DNA fragmentation and also biochemical phenomena such as the activation of caspases which cause damage to key structural components of the cell, so inducing its disassembly and death. Regulation of the process of apoptosis is complex and involves the activation or repression of several intracellular signalling pathways (Cory S. et al., Nature Review Cancer, 2002, 2, 647-656).

Deregulation of apoptosis is involved in certain pathologies. Increased apoptosis is associated with neurodegenerative diseases such as Parkinson's disease, Alzheimer's disease and ischaemia. Conversely, deficits in the implementation of apoptosis play a significant role in the development of cancers and their chemoresistance, in auto-immune diseases, inflammatory diseases and viral infections. Accordingly, absence of apoptosis is one of the phenotypic signatures of cancer (Hanahan D. et al., Cell 2000, 100, 57-70).

In addition to being new, the compounds of the present invention have pro-apoptotic properties making it possible to use them in pathologies involving a defect in apoptosis, such as, for example, in the treatment of cancer.

The present invention relates more especially to compounds of formula (I):

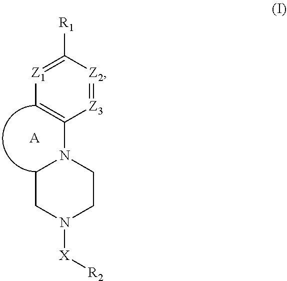

wherein:
A represents a 5, 6 or 7-membered aromatic or non-aromatic ring which may contain 1 or 2 hetero atoms selected from oxygen, sulphur and nitrogen, it being possible for the latter to be substituted by a linear or branched (C₁-C₆)alkyl group, it being understood that the ring A so defined cannot contain 2 sulphur atoms or 2 oxygen atoms and that one of the ring members may be a C═O group,
Z₁, Z₂ and Z₃ each independently of the others represents a CH group or a nitrogen atom, it being understood that at least one of these three groups is a nitrogen atom,
R₂ represents an aryl or heteroaryl group,
X represents a linear or branched alkylene chain containing from 1 to 6 carbon atoms, one or two of the carbon atoms of which may be replaced by an oxygen atom, a cycloalkylene group, an arylene group, a heteroarylene group or an SO₂ group,
R₁ represents a group of formula (II):

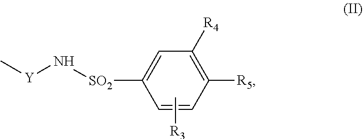

wherein:
Y represents a group C═O or CH₂,
R₄ represents a hydrogen atom, in which case R₅ represents a hydrogen atom or a group —NR₆R′₆ or —CH₂—NR₆R′₆ wherein each of R₆ and R′₆, which may be the same or different, independently of the other represents a hydrogen atom or a linear or branched (C₁-C₆)alkyl group substituted by one or more aryl, heteroaryl, aryloxy, heteroaryloxy, arylthio, heteroarylthio, heterocycloalkyl or —NR₉R′₉ groups wherein:
   R₉ and R′₉, which may be the same or different, are selected from hydrogen, linear or branched (C₁-C₆)alkyl, linear or branched (C₁-C₆)alkoxy, aryl and heteroaryl,
   or R₉ and R′₉ form a saturated or unsaturated cyclic or bicyclic group optionally substituted by a hetero atom selected from oxygen, nitrogen and sulphur, it being understood that one or more of the ring members may represent a C═O group or may be substituted as indicated in the definition of a heterocycle given hereinafter,

or R₄ and R₅, together with the two carbon atoms carrying them, form an aromatic or non-aromatic ring containing 5 or 6 ring members, including a nitrogen atom in the position para to the SO₂ group, which in addition to the nitrogen atom may contain a further nitrogen atom and/or an SO₂ group, the ring so defined being substituted by an R₆ group as defined hereinbefore,
R₃ represents a halogen atom or an NO₂, R₇, SO₂—R₈, linear or branched (C₁-C₆)alkyl or linear or branched (C₁-C₆)alkoxy group, wherein R₇ may take any of the meanings of R₆ as defined hereinbefore,
R₈ represents an amino group or a linear or branched (C₁-C₆)alkyl group optionally substituted by one or more halogen atoms,

it being understood that:
“aryl” means a phenyl, naphthyl or biphenyl group,
“heteroaryl” means any mono- or bi-cyclic group having at least one aromatic moiety and containing from 5 to 10 ring members, which may contain from 1 to 3 hetero atoms selected from oxygen, sulphur and nitrogen, such as the groups furan, thiophene, pyrrole, imidazoline, pyridine, quinoline, isoquinoline, chroman, indole, benzothiophene, benzofuran, 1,3-benzodioxole and 2,3-dihydro-1,4-benzodioxine,
“heterocycloalkyl” means any mono- or bi-cyclic non-aromatic group containing from 4 to 10 ring members, which may contain from 1 to 3 hetero atoms selected from oxygen, sulphur and nitrogen,
“cycloalkyl” means any mono- or bi-cyclic non-aromatic group containing from 4 to 10 ring members,

it, being possible for the aryl, heteroaryl, heterocycloalkyl and cycloalkyl groups so defined to be substituted by from 1 to 3 groups selected from linear or branched (C₁-C₆)alkyl optionally substituted by a hydroxy or amino group, linear or branched (C₁-C₆)alkoxy, hydroxy, carboxy, formyl, nitro, cyano, amino, linear or branched (C₁-C₆)polyhaloalkyl, alkyloxycarbonyl and halogen atoms,
“arylene”, “heteroarylene” and “cycloalkylene” mean, respectively, an aryl, heteroaryl and cycloalkyl group as defined above, inserted in the place of a carbon atom of the alkylene chain,

to their enantiomers and diastereoisomers, and to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Y advantageously represents a C═O group.

Preference is given to the R₃ group being NO₂.

Preferred X—R₂ groups are ([1,1′-biphenyl]-2-yl)methyl groups optionally substituted by one or more halogen atoms.

R₄ preferably represents a hydrogen atom.

Preference is given to the R₆ group being the group 1-(N,N-dimethylamino)-4-(phenylsulphanyl)-butan-3-yl.

R′₆ advantageously represents a hydrogen atom.

The invention relates even more especially to the compounds of formula (I) which are:
N-({2-[(4′-chloro[1,1′-biphenyl]-2-yl)methyl]-1,2,3,4,10,10a-hexahydropyrido-[4′,3′:4,5]pyrrolo[1,2-a]pyrazin-8-yl}carbonyl)-4-({(1R)-3-(dimethylamino)-1-[(phenylsulphanyl)methyl]propyl}amino)-3-nitrobenzenesulphonamide,
N-({8-[(4′-chloro[1,1′-biphenyl]-2-yl)methyl]-6,7,8,9,9a,10-hexahydropyrido-[2′,3′:4,5]pyrrolo[1,2-a]pyrazin-2-yl}carbonyl)-4-({(1R)-3-(dimethylamino)-1-[(phenylsulphanyl)methyl]propyl}amino)-3-nitrobenzenesulphonamide,
N-({7-[(4′-chloro[1,1′-biphenyl]-2-yl)methyl]-5,5a,6,7,8,9-hexahydropyrido-[3′,2′:4,5]pyrrolo[1,2-a]pyrazin-3-yl}carbonyl)-4-({(1R)-3-(dimethylamino)-1-[(phenylsulphanyl)methyl]propyl}amino)-3-nitrobenzenesulphonamide,
N-({3-[(4′-chloro[1,1′-biphenyl]-2-yl)methyl]-2,3,4,4a,5,6-hexahydro-1H-pyrazino[1,2-a][1,5]naphthyridin-8-yl}carbonyl)-4-({(1R)-3-(dimethylamino)-1-[(phenylsulphanyl)methyl]propyl}amino)-3-nitrobenzenesulphonamide,
N-({8-[(4′-chloro[1,1′-biphenyl]-2-yl)methyl]-6,6a,7,8,9,10-hexahydro-5H-pyrazino[1,2-a][1,7]naphthyridin-3-yl}carbonyl)-4-({(1R)-3-(dimethylamino)-1-[(phenylsulphanyl)methyl]propyl}amino)-3-nitrobenzenesulphonamide,
N-({8-[(4′-chloro[1,1′-biphenyl]-2-yl)methyl]-6,6a,7,8,9,10-hexahydro-5H-pyrazino[1,2-a][1,8]naphthyridin-3-yl}carbonyl)-4-({(1R)-3-(dimethylamino)-1-[(phenylsulphanyl)methyl]propyl}amino)-3-nitrobenzenesulphonamide.

The enantiomers and diastereoisomers of the preferred compounds of the invention and also addition salts thereof with a pharmaceutically acceptable acid or base form an integral part of the invention.

The invention relates also to a process for the preparation of compounds of formula (I), which process is characterised in that there is used as starting material the compound of formula (III):

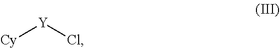

wherein Y is as defined for formula (I) and Cy represents a fused tricyclic system of formula (IV):

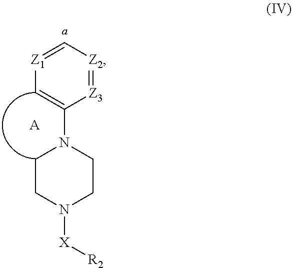

wherein A, X, Z₁, Z₂, Z₃ and R₂ are as defined for formula (I), the —Y—Cl group being attached in the a position of the tricyclic system so defined,

which compound of formula (III) is condensed, in a basic medium in the presence or absence of a coupling agent, with the compound of formula (V):

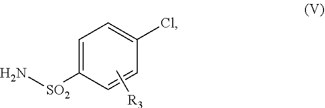

wherein R₃ is as defined for formula (I),

to obtain the compound of formula (VI):

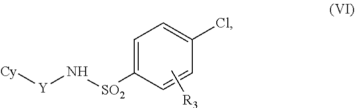

wherein Cy, Y and R₃ are as defined hereinbefore,

which is condensed with the compound of formula HNR₆R′₆, wherein R₆ and R′₆ are as defined for formula (I), to yield the compound of formula (I/a), a particular case of the compounds of formula (I):

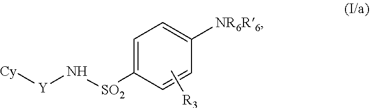

wherein Cy, Y, R₃, R₆ and R′₆ are as defined hereinbefore,

which may be purified according to a conventional separation technique, which is converted, if desired, into its addition salts with a pharmaceutically acceptable acid or base and which is optionally separated into its isomers according to a conventional separation technique.

The compounds of formulae (III) and (V) are either commercially available or can be obtained by the person skilled in the art using conventional chemical reactions described in the literature.

An advantageous variant relates to a process for the preparation of compounds of formula (I), which process is characterised in that there is used as starting material the compound of formula (III′):

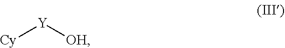

wherein Y is as defined for formula (I) and Cy represents a fused tricyclic system of formula (IV):

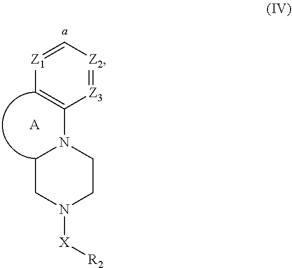

wherein A, X, Z₁, Z₂, Z₃ and R₂ are as defined for formula (I), the —Y—OH group being attached in the a position of the tricyclic system so defined,

which compound of formula (III′) is condensed, in a basic medium in the presence of a coupling agent, with the compound of formula (VII):

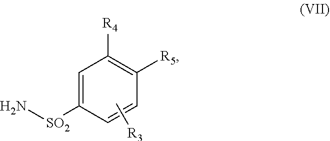

wherein R₃, R₄ and R₅ are as defined for formula (I),

to yield the compound of formula (I), which may be purified according to a conventional separation technique, which is converted, if desired, into its addition salts with a pharmaceutically acceptable acid or base and which is optionally separated into its isomers according to a conventional separation technique.

The compounds of formulae (III′) and (VII) are either commercially available or can be obtained by the person skilled in the art using conventional chemical reactions described in the literature.

Pharmacological study of the compounds of the invention has shown that they have pro-apoptotic properties. The ability to reactivate the apoptotic process in cancerous cells is of major therapeutic interest in the treatment of cancers.

More especially, the compounds according to the invention will be useful in the treatment of chemo- or radio-resistant cancers, and in malignant haemopathies and small-cell lung cancer.

Among the cancer treatments envisaged there may be mentioned, without implying any limitation, cancers of the bladder, brain, breast and uterus, chronic lymphoid leukaemias, cancers of the colon, oesophagus and liver, lymphoblastic leukaemias, follicular lymphomas, melanomas, malignant haemopathies, myelomas, ovarian cancers, non-small-cell lung cancers, prostate cancers and small-cell lung cancers.

The present invention relates also to pharmaceutical compositions comprising at least one compound of formula (I) on its own or in combination with one or more pharmaceutically acceptable excipients.

Among the pharmaceutical compositions according to the invention, there may be mentioned more especially those that are suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels, and drinkable or injectable ampoules.

The useful dosage varies according to the sex, age and weight of the patient, the administration route, the nature of the therapeutic indication, and any associated treatments, and ranges from 0.01 mg to 1 g per 24 hours in one or more administrations.

Furthermore, the present invention relates also to the association of a compound of formula (I) with an anticancer agent selected from genotoxic agents, mitotic poisons, anti-metabolites, proteasome inhibitors and kinase inhibitors, and also to use of that type of association in the manufacture of medicaments for use in the treatment of cancer.

The compounds of the invention may also be used in association with radiotherapy in the treatment of cancer.

The following Preparations and Examples illustrate the invention but do not limit it in any way.

### PREPARATION 1

### 2-[(4′-Chloro[1,1′-biphenyl]-2-yl)methyl]-1,2,3,4,10,10a-hexahydropyrido[4′,3′:4,5]pyrrolo[1,2-a]pyrazine-8-carboxylic acid tris(trifluoroacetate)

### Step A: 2-Methoxy-4-methyl-5-nitropyridine

To 45 mL of a solution of sodium methanolate in methanol (30% by weight) in 30 mL of methanol there is added, in portions, over 45 minutes, 2-chloro-4-methyl-5-nitropyridine (25 g). The reaction mixture thereby obtained is stirred for 4 hours and then for 1 hour at reflux before being poured over 1 L of ice-cold water. The heterogeneous mixture is stirred overnight and then filtered to yield, after drying, the title product in the form of a chestnut-brown solid.

### Step B: Ethyl 3-(2-methoxy-5-nitro-4-pyridyl)-2-oxopropanoate

To 30 mL of ethanol there are added potassium tert-butylate (15.14 g), diethyl ether (300 mL) and diethyl oxalate (18.43 mL). After being in contact for 15 minutes, the compound of Step A (22.69 g) is added, and then the reaction mixture thereby obtained is stirred at reflux for 4 hours and at ambient temperature overnight. The red precipitate obtained is filtered off and rinsed with diethyl ether and then diluted with 500 mL of water. Glacial acetic acid is added until a pH of 4 is obtained; the reaction mixture is then stirred at ambient temperature for 2 hours and then filtered to yield the title product in the form of a beige solid.

### Step C: Ethyl 5-methoxy-1H-pyrrolo[2,3-c]pyridine-2-carboxylate

To a heterogeneous solution of the compound of Step B (27.84 g) in 350 mL of ethanol there is added palladium-on-carbon (5.9 g, 20% by weight). The reaction mixture is hydrogenated for 24 hours and then filtered and concentrated. The residue obtained is taken up in a mixture of ether/water; the product is then extracted using ether. The organic phases are washed with saturated aqueous potassium carbonate solution, dried over magnesium sulphate, filtered and concentrated to yield the title product in the form of a brown solid.

### Step D: Ethyl 1-acetyl-5-methoxy-2,3-dihydro-1H-pyrrolo[2,3-c]pyridine-2-carboxylate

To a solution of 10 g of the compound of Step C in 60 mL of dichloromethane there are added triethylamine (7.57 mL), 4-dimethylaminopyridine (550 mg) and, dropwise, acetic anhydride (5.48 mL). The reaction mixture is stirred for 24 hours at ambient temperature and then diluted with water. The product is extracted using dichloromethane; the organic phases are then washed with water and using saturated aqueous sodium chloride solution. They are then dried over magnesium sulphate, filtered and concentrated.

To a solution, in 200 mL of ethanol, of the residue thereby obtained, there is added palladium(II) chloride (798 mg). The reaction mixture is then hydrogenated for 24 hours. After filtration and concentration, the residue is purified by flash chromatography over silica gel (heptane/ethyl acetate) to yield the title product in the form of an off-white solid.

### Step E: Ethyl 5-methoxy-2,3-dihydro-1H-pyrrolo[2,3-c]pyridine-2-carboxylate

A solution of the protected azaindoline of the Step above (17.9 g) in 75 mL of 1N ethanolic HCl is placed under reflux for 8 hours. After returning to ambient temperature, the reaction mixture is neutralised with saturated aqueous sodium hydrogen carbonate solution; the product is then extracted using ethyl acetate. The organic phase is concentrated and the residue is used directly in the next Step.

### Step F: 8-Methoxy-2,3,10,10a-tetrahydropyrido[4′,3′:4,5]pyrrolo[1,2-a]pyrazine-1,4-dione

To a solution of benzyloxycarbonylaminoacetic acid (17 g) in 300 mL of tetrahydrofuran there is added at 0° C., in portions, PCl₅ (15 g). The reaction mixture is stirred at that same temperature for 2 hours; there is then added thereto, dropwise, a solution of the compound of Step E in 100 mL of tetrahydrofuran and 50 mL of pyridine. After returning to ambient temperature, the reaction mixture is stirred for 12 hours. The reaction mixture is hydrolysed at 0° C., dropwise, and then extracted using ethyl acetate. The organic phases are washed with saturated aqueous sodium hydrogen carbonate solution and saturated aqueous sodium chloride solution, dried over magnesium sulphate, filtered and concentrated. After filtration over silica, the solid obtained is dissolved in 100 mL of tetrahydrofuran and 350 mL of methanol. Ammonium formate (4.8 g) is then added in portions, and the reaction mixture is placed under reflux for 10 hours. After filtration and rinsing with warm tetrahydrofuran and warm dimethylformamide, the filtrate is concentrated and taken up in cold methanol to yield the title product in the form of a white solid.

### Step G: 2-Benzyl-8-methoxy-2,3,10,10a-tetrahydropyrido[4′,3′:4,5]pyrrolo[1,2-a]-pyrazine-1,4-dione

To a heterogeneous solution of sodium hydride (1.82 g) in 50 mL of dimethylformamide there is added at 0° C., dropwise, a solution of the compound of Step F (7.09 g) in 450 mL of dimethylformamide over a period of 2 hours. After returning to ambient temperature over 4 hours, benzyl bromide (4.54 mL) is added over 25 minutes. The reaction mixture is stirred for 17 hours and is then concentrated. The residue obtained is taken up in a mixture of ethyl acetate and water, and the product is extracted using ethyl acetate. The organic phases are washed with saturated aqueous lithium chloride solution, dried over magnesium sulphate, filtered and concentrated. After purification by flash chromatography over silica gel (heptane/ethyl acetate), the title product is obtained in the form of an off-white solid.

### Step H: 2-Benzyl-8-methoxy-1,2,3,4,10,10a-hexahydropyrido[4′,3′:4,5]pyrrolo-[1,2-a]pyrazine

To a solution of the compound of Step G (7.72 g) in 225 mL of tetrahydrofuran and 225 mL of toluene there is added at 0° C., dropwise over 50 minutes, a 1M solution of lithium aluminium hydride in tetrahydrofuran (75 mL). The reaction mixture is placed under reflux for 6 hours. After returning to ambient temperature, it is hydrolysed dropwise at 0° C. by respective additions of 10 mL of water, 10 mL of 15% aqueous sodium hydroxide solution and 30 mL of water. After returning to ambient temperature, the mixture is stirred vigorously for 6 hours and then filtered. The filtrate is concentrated and taken up in a mixture of ethyl acetate and saturated aqueous sodium hydrogen carbonate solution. The product is then extracted using ethyl acetate. After concentrating the organic phases, the residue is purified by flash chromatography over silica gel (dichloromethane/methanol) to yield the title product in the form of a brown oil.

### Step I: 2-Benzyl-1,2,3,4,10,10a-hexahydropyrido[4′,3′:4,5]pyrrolo[1,2-a]pyrazin-8-yl trifluoromethanesulphonate

A solution of the compound of Step H (2.6 g) in a 33% solution of hydrobromic acid in acetic acid is heated at reflux for 3 hours. After returning to ambient temperature, aqueous sodium hydrogen carbonate solution is added at 0° C., dropwise, until a pH of 8 is obtained. The product is extracted using dichloromethane, and then the organic phases are combined, dried over magnesium sulphate, filtered and concentrated. To a solution, in 70 mL of pyridine, of the residue thereby obtained there is added at 0° C., dropwise over 50 minutes, triflic anhydride (13 mL). The reaction mixture is brought back to ambient temperature over 16 hours and is then concentrated. The residue is taken up in a mixture of ethyl acetate and water. The product is extracted using ethyl acetate; the organic phases are then washed with aqueous sodium hydrogen carbonate solution, dried over magnesium sulphate, filtered and concentrated. The residue is purified by flash chromatography over silica gel (heptane/ethyl acetate) to yield the title product in the form of a green oil.

### Step J: Methyl 2-benzyl-1,2,3,4,10,10a-hexahydropyrido[4′,3′:4,5]pyrrolo[1,2-a]-pyrazine-8-carboxylate

To a solution of the triflate compound of the Step above (2.3 g) in 58 mL of a mixture of dimethyl sulphoxide/methanol (3/2) there are added triethylamine (1.8 mL), 1,1′-bis(di-phenylphosphino)ferrocene (643 mg) and palladium(II) diacetate (130.2 mg). The reaction mixture is subjected to bubbling-in of argon over 15 mins. and then of carbon monoxide. The flask is then pressurised under carbon monoxide. The reaction mixture is heated to 65° C. and then stirred for 18 hours. After concentration, the residue is taken up in a mixture of ethyl acetate/water. The product is extracted with ethyl acetate; the organic phases are then washed with saturated aqueous sodium hydrogen carbonate solution, dried over magnesium sulphate, filtered and concentrated. After lyophilisation in dioxane, the residue is purified by flash chromatography over silica gel (dichloromethane/methanol) to yield the title product.

### Step K: Methyl 1,2,3,4,10,10a-hexahydropyrido[4′,3′:4,5]pyrrolo[1,2-a]pyrazine-8-carboxylate dihydrochloride

To a solution of the compound of the Step above (1.49 g) in 35 mL of 1N methanolic HCl solution there is added palladium(II) hydroxide on carbon (300 mg, 20% by weight). The reaction mixture is hydrogenated over 16 hours and then filtered and concentrated to yield the title product in the form of a brown solid.

### Step L: Methyl 2-[(4′-chloro[1,1′-biphenyl]-2-yl)methyl]-1,2,3,4,10,10a-hexahydropyrido[4′,3′:4,5]pyrrolo[1,2-a]pyrazine-8-carboxylate

To a solution of the compound of Step K (1.016 g) in 15 mL of dimethylformamide there are added triethylamine (1.84 mL), 4′-chloro-2-chloromethylbiphenyl (787 mg) and sodium iodide (50 mg). The reaction mixture is stirred at 50° C. for 3 hours, concentrated and then taken up in a mixture of ethyl acetate and saturated aqueous sodium hydrogen carbonate solution. After extraction using ethyl acetate, the organic phases are washed with saturated lithium chloride solution, dried over magnesium sulphate, filtered and concentrated. The residue is purified by flash chromatography over silica gel (heptane/ethyl acetate) to yield the title product in the form of an off-white solid.

### Step M: 2-[(4′-Chloro[1,1′-biphenyl]-2-yl)methyl]-1,2,3,4,10,10a-hexahydropyrido-[4′,3′:4,5]pyrrolo[1,2-a]pyrazine-8-carboxylic acid tris(trifluoroacetate)

To a suspension of the ester compound of the Step above (1.085 g) in a mixture of dioxane/water (4/1) there is added lithium hydroxide (525 mg). The reaction mixture is stirred at ambient temperature for 24 hours and is then concentrated and taken up in 20 mL of a mixture of water and 1N aqueous hydrochloric acid solution (1/1). The reaction mixture is stirred for 2 hours at ambient temperature and then concentrated. The residue is purified on an Oasis cartridge (water/acetonitrile/trifluoroacetic acid) and lyophilised in a mixture of acetonitrile and water to yield the title product.

### PREPARATION 2

### 8-[(4′-Chloro[1,1′-biphenyl]-2-yl)methyl]-6,7,8,9,9a,10-hexahydropyrido[2′,3′:4,5]pyrrolo[1,2-a]pyrazine-2-carboxylic acid trifluoroacetate

The procedure is as in Steps A to M of Preparation 1, replacing the 2-chloro-4-methyl-5-nitropyridine in Step A by 6-chloro-2-methyl-3-nitropyridine.

### PREPARATION 3

### 7-[(4′-Chloro[1,1′-biphenyl]-2-yl)methyl]-5,5a,6,7,8,9-hexahydropyrido[3′,2′:4,5]pyrrolo[1,2-a]pyrazine-3-carboxylic acid trifluoroacetate

### Step A: tert-Butyl 3-methyl-2-pyridylcarbamate

To a solution of di-tert-butyldicarbonate (161.5 g) in 400 mL of hexane there is added, over 2 hours, a solution of 3-methyl-2-aminopyridine (50 g) in 50 mL of ethyl acetate. The reaction mixture is heated at reflux for 1 hour, and then diluted with 550 mL of hexane and stirred at ambient temperature for 2 hours. The precipitate is filtered off to yield the title product in the form of a white solid.

### Step B: Ethyl 3-{2-[(tert-butoxycarbonyl)amino]-3-pyridyl}-2-oxopropanoate

To a solution of the compound of the Step above (10 g) in 200 mL of tetrahydrofuran there is added, at a temperature below 5° C., n-butyllithium. The reaction mixture is then stirred at 0° C. for 1 hour 15 minutes before being transferred by cannula, at a temperature below −3° C., to a solution of diethyl oxalate (14 mL) in 50 mL of tetrahydrofuran.

### Step C: Ethyl 1H-pyrrolo[2,3-b]pyridine-2-carboxylate

The reaction mixture of the Step above is stirred at ambient temperature for 2 hours 15 minutes and then gently poured into 30 mL of 5N hydrochloric acid solution at a temperature not exceeding 10° C. The solution obtained is placed under reflux for 2 hours. After returning to ambient temperature, it is neutralised until the pH is 3, using 5N sodium hydroxide solution. The product is extracted using ether; the organic phases are then washed with saturated potassium carbonate solution, dried over magnesium sulphate, filtered and concentrated. The residue is taken up in methanol to yield the title product in the form of a beige solid.

### Step D: Ethyl 1-acetyl-2,3-dihydro-1H-pyrrolo[2,3-b]pyridine-2-carboxylate

The procedure described in Step D of Preparation 1 is applied to the azaindole obtained in the Step above. The title product is ultimately in the form of a light-green solid.

### Step E: 5,5a,7,8-Tetrahydropyrido[3′,2′:4,5]pyrrolo[1,2-a]pyrazine-6,9-dione

The procedure described in Step E of Preparation 1 is applied to the azaindoline obtained in the Step above. The title product is ultimately in the form of a white solid.

### Step F: 7-Benzyl-5,5a,7,8-tetrahydropyrido[3′,2′:4,5]pyrrolo[1,2-a]pyrazine-6,9-dione

The procedure described in Step F of Preparation 1 is applied to the compound of the Step above. The title product is ultimately in the form of a pale yellow solid.

### Step G: 7-Benzyl-5,5a,6,7,8,9-hexahydropyrido[3′,2′:4,5]pyrrolo[1,2-a]pyrazine

The procedure described in Step G of Preparation 1 is applied to the compound of the Step above. The title product is ultimately in the form of a brown oil.

### Step H: 7-Benzyl-3-bromo-5,5a,6,7,8,9-hexahydropyrido[3′,2′:4,5]pyrrolo[1,2-a]-pyrazine

To a solution of the compound of Step G in 50 mL of dimethylformamide there is added at 0° C., dropwise over 45 minutes, a solution of N-bromosuccinimide (785 mg) in 30 mL of dimethylformamide. The reaction mixture is stirred at that same temperature for 2.5 hours before being hydrolysed. The product is then extracted using ethyl acetate. The organic phases are washed with saturated lithium chloride solution, dried over magnesium sulphate, filtered and concentrated. The residue is purified by flash chromatography over silica gel (heptane/ethyl acetate) to obtain the title product in the form of a brown oil.

### Step I: Methyl 7-benzyl-5,5a,6,7,8,9-hexahydropyrido[3′,2′:4,5]pyrrolo[1,2-a]pyrazine-3-carboxylate

The procedure described in Step J of Preparation 1 is applied to the compound of the Step above. The title product is ultimately in the form of a brown oil.

### Step J: Methyl 5,5a,6,7,8,9-hexahydropyrido[3′,2′:4,5]pyrrolo[1,2-a]pyrazine-3-carboxylate dihydrochloride

The procedure described in Step K of Preparation 1 is applied to the compound of the Step above. The title product is ultimately in the form of a brown solid.

### Step K: Methyl 7-[(4′-chloro[1,1′-biphenyl]-2-yl)methyl]-5,5a,6,7,8,9-hexahydropyrido[3′,2′:4,5]pyrrolo[1,2-a]pyrazine-3-carboxylate

The procedure described in Step L of Preparation 1 is applied to the compound of the Step above. The title product is ultimately in the form of a white solid.

### Step L: 7-[(4′-Chloro[1,1′-biphenyl]-2-yl)methyl]-5,5a,6,7,8,9-hexahydropyrido-[3′,2′:4,5]pyrrolo[1,2-a]pyrazine-3-carboxylic acid trifluoroacetate

The procedure described in Step M of Preparation 1 is applied to the compound of the Step above. The title product is ultimately in the form of a white solid.

### PREPARATION 4

### 3-[(4′-Chloro[1,1′-biphenyl]-2-yl)methyl]-2,3,4,4a,5,6-hexahydro-1H-pyrazino[1,2-a][1,5]naphthyridine-8-carboxylic acid trifluoroacetate

The operations described in Steps F to M of Preparation 1 are repeated, in Step F replacing the compound of Step E with methyl 6-methoxy-1,2,3,4-tetrahydro[1,5]naphthyridine-2-carboxylate.

### PREPARATION 5

### 8-[(4′-Chloro[1,1′-biphenyl]-2-yl)methyl]-6,6a,7,8,9,10-hexahydro-5H-pyrazino[1,2-a][1,7]naphthyridine-3-carboxylic acid trifluoroacetate

The operations described in Steps F to M of Preparation 1 are repeated, in Step F replacing the compound of Step E with methyl 6-methoxy-1,2,3,4-tetrahydro[1,7]naphthyridine-2-carboxylate.

### PREPARATION 6

### 8-[(4′-Chloro[1,1′-biphenyl]-2-yl)methyl]-6,6a,7,8,9,10-hexahydro-5H-pyrazino[1,2-a][1,8]naphthyridine-3-carboxylic acid trifluoroacetate

The operations described in Steps F to M of Preparation 1 are repeated, in Step F replacing the compound of Step E with methyl 6-methoxy-1,2,3,4-tetrahydro[1,8]naphthyridine-2-carboxylate.

### EXAMPLE 1

### N-({2-[(4′-Chloro[1,1′-biphenyl]-2-yl)methyl]-1,2,3,4,10,10a-hexahydropyrido[4′,3′:4,5]pyrrolo[1,2-a]pyrazin-8-yl}carbonyl)-4-({(1R)-3-(dimethylamino)-1-[(phenylsulphanyl)methyl]propyl}amino)-3-nitro-benzenesulphonamide trihydrochloride

### Step A: N-({2-[(4′-Chloro[1,1′-biphenyl]-2-yl)methyl]-1,2,3,4,10,10a-hexahydropyrido[4′,3′:4,5]pyrrolo[1,2-a]pyrazin-8-yl}carbonyl)-4-({(1R)-3-(dimethyl-amino)-1-[(phenylsulphanyl)methyl]propyl}amino)-3-nitrobenzene-sulphonamide

To a solution of the compound of Preparation 1 (346 mg) in 10 mL of dichloromethane there are added diisopropylethylamine (300 μL), 4-({(1R)-3-(dimethylamino)-1-[(phenylsulphanyl)methyl]propyl}amino)-3-nitrobenzenesulphonamide (191 mg), N-ethyl-N′-3-dimethylaminopropylcarbodiimine (121 mg) and 4-dimethylaminopyridine (77 mg, 0.63 mmol, 1.4 eq.). The reaction mixture is stirred at ambient temperature for 6 days and then hydrolysed using saturated aqueous ammonium chloride solution. The product is extracted using ethyl acetate. The organic phases are dried over magnesium sulphate, filtered and concentrated. The residue is purified by flash chromatography over silica gel (dichloromethane/ammoniacal methanol) to yield the title product in the form of a pale yellow solid.

### Step B: N-({2-[(4′-Chloro[1,1′-biphenyl]-2-yl)methyl]-1,2,3,4,10,10a-hexahydropyrido[4′,3′:4,5]pyrrolo[1,2-a]pyrazin-8-yl}carbonyl)-4-({(1R)-3-(dimethyl-amino)-1-[(phenylsulphanyl)methyl]propyl}amino)-3-nitrobenzene-sulphonamide trihydrochloride

The compound of Step A is dissolved in 4 mL of dichloromethane and then 1N ethereal HCl solution (805 μL) is added. The reaction mixture is stirred at ambient temperature for 2 hours and then concentrated to yield the title product in the form of a yellow solid after lyophilisation.

Elemental Microanalysis:

| | % C | % H | % N | % S | % Cl |
|---|---|---|---|---|---|
| Calculated | 50.66 | 5.56 | 9.85 | 6.44 | 12.80 |
| Found | 50.43 | 5.38 | 10.02 | 6.37 | 12.93 |

### EXAMPLE 2

### N-({8-[(4′-Chloro[1,1′-biphenyl]-2-yl)methyl]-6,7,8,9,9a,10-hexahydropyrido[2′,3′:4,5]pyrrolo[1,2-a]pyrazin-2-yl}carbonyl)-4-({(1R)-3-(dimethylamino)-1-[(phenylsulphanyl)methyl]propyl}amino)-3-nitro-benzenesulphonamide dihydrochloride

The procedure is as in Steps A and B of Example 1, in Step A replacing the compound of Preparation 1 by the compound of Preparation 2.

Elemental Microanalysis:

| | % C | % H | % N | % S | % Cl |
|---|---|---|---|---|---|
| Calculated | 54.40 | 5.27 | 10.57 | 6.91 | 12.23 |
| Found | 54.73 | 5.21 | 10.45 | 6.45 | 12.49 |

### EXAMPLE 3

### N-({7-[(4′-Chloro[1,1′-biphenyl]-2-yl)methyl]-5,5a,6,7,8,9-hexahydropyrido[3′,2′:4,5]pyrrolo[1,2-a]pyrazin-3-yl}carbonyl)-4-({(1R)-3-(dimethylamino)-1-[(phenylsulphanyl)methyl]propyl}amino)-3-nitro-benzenesulphonamide trihydrochloride

The procedure is as in Steps A and B of Example 1, in Step A replacing the compound of Preparation 1 by the compound of Preparation 3.

Elemental Microanalysis:

| | % C | % H | % N | % S | % Cl |
|---|---|---|---|---|---|
| Calculated | 53.50 | 5.21 | 10.40 | 6.80 | 13.91 |
| Found | 53.82 | 5.24 | 10.20 | 6.74 | 14.13 |

### EXAMPLE 4

### N-({3-[(4′-Chloro[1,1′-biphenyl]-2-yl)methyl]-2,3,4,4a,5,6-hexahydro-1H-pyrazino[1,2-a][1,5]naphthyridin-8-yl}carbonyl)-4-({(1R)-3-(dimethyl-amino)-1-[(phenylsulphanyl)methyl]propyl}amino)-3-nitrobenzene-sulphonamide dihydrochloride

The procedure is as in Steps A and B of Example 1, in Step A replacing the compound of Preparation 1 by the compound of Preparation 4.

### EXAMPLE 5

### N-({8-[(4′-Chloro[1,1′-biphenyl]-2-yl)methyl]-6,6a,7,8,9,10-hexahydro-5H-pyrazino[1,2-a][1,7]naphthyridin-3-yl}carbonyl)-4-({(1R)-3-(dimethylamino)-1-[(phenylsulphanyl)methyl]propyl}amino)-3-nitro-benzenesulphonamide dihydrochloride

The procedure is as in Steps A and B of Example 1, in Step A replacing the compound of Preparation 1 by the compound of Preparation 5.

### EXAMPLE 6

### N-({8-[(4′-Chloro[1,1′-biphenyl]-2-yl)methyl]-6,6a,7,8,9,10-hexahydro-5H-pyrazino[1,2-a][1,8]naphthyridin-3-yl}carbonyl)-4-({(1R)-3-(dimethylamino)-1-[(phenylsulphanyl)methyl]propyl}amino)-3-nitrobenzenesulphonamide dihydrochloride

The procedure is as in Steps A and B of Example 1, in Step A replacing the compound of Preparation 1 by the compound of Preparation 6.

### Pharmacological Study

### Example A

### Induction of Caspase Activity In Vitro

This study was carried out on the following human tumour cell line:
1 small-cell lung carcinoma, H146.

This cell line is cultured in an incubator at 37° C. in the presence of 5% CO₂. More specifically, the H146 cells are cultured in complete RPMI 1640 medium containing 10% foetal calf serum, 2 mM glutamine, 50 units/ml of penicillin, 50 μg/ml of streptomycin and 10 mM Hepes buffer, pH=7.4.

The cells are distributed onto 6-well plates and exposed to the test compounds for 6 hours. They are then collected and lysed and the caspase activity is measured in the cell lysates. This enzymatic measurement is carried out by measuring the appearance of a fluorigenic cleavage product (Pharmacia).

The results show that the compounds of the invention are powerful apoptosis inducers, evaluated by measuring caspase 3 activity in the tumour line tested.

By way of example, the compounds of Examples 1 to 3 at 10 μM show activities of, respectively, 15395, 9948 and 17776 RFU (Relative Fluorescence Units).

### Example B

### Cytotoxicity In Vitro

The cytotoxicity studies were carried out on the tumour line given in Example A.

The cells are distributed onto microplates and exposed to the test compounds for 48 hours. The cell viability is then quantified by a colorimetric assay, the Microculture Tetrazolium Assay (Cancer Res., 1987, 47, 939-942).

The results are expressed in IC₅₀ (the concentration of compound that inhibits cell viability by 50%) and they show that the compounds of the invention are cytotoxic.

### Example C

### Induction of Caspase Activity In Vivo

The ability of the compounds of the invention to activate caspase 3 is evaluated in a xenograft model of H146 small-cell lung carcinoma cells.

5×10⁶ H146 cells are grafted sub-cutaneously into immunosuppressed mice (NOD SCID strain). 25 to 30 days after the graft, the test compounds are injected by the intra-peritoneal route in a mixture of Tween 80/water. Sixteen hours after treatment, the tumour masses are recovered and lysed, and the caspase 3 activity is measured in the tumour lysates.

The results obtained show that the compounds of the invention are capable of inducing apoptosis in H146 tumour lines in vivo.

### Example D

### Pharmaceutical Composition: Tablets

| | | |
|---|---|---|
| 1000 tablets containing a dose of 5 mg of N-({2-[(4′-chloro[1,1′- | 5 | g |
| biphenyl]-2-yl)methyl]-1,2,3,4,10,10a- | | |
| hexahydropyrido[4′,31:4,5]pyrrolo[1,2-a]pyrazin-8- | | |
| yl}carbonyl)-4-({(1R)-3-(dimethylamino)-1- | | |
| [(phenylsulphanyl)methyl]propyl}amino)- | | |
| 3-nitrobenzenesulphonamide trihydrochloride (Example 1) | | |
| Wheat starch | 20 | g |
| Maize starch | 20 | g |
| Lactose | 30 | g |
| Magnesium stearate | 2 | g |
| Silica | 1 | g |
| Hydroxypropylcellulose | 2 | g |

1-17. (canceled)

18- A compound selected from those of formula (I): 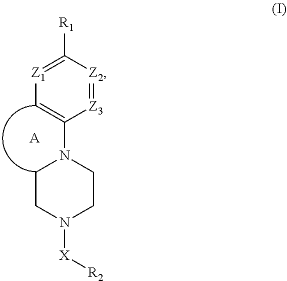
wherein:
A represents a 5, 6 or 7-membered aromatic or non-aromatic ring having 1 or 2 hetero atoms selected from oxygen, sulphur and nitrogen, wherein the nitrogen atom may optionally be substituted by a linear or branched (C₁-C₆)alkyl group, it being understood that ring A cannot contain 2 sulphur atoms or 2 oxygen atoms and that one of the ring members may be a C═O group,
Z₁, Z₂ and Z₃ each independently represents a CH group or a nitrogen atom, it being understood that at least one of Z₁, Z₂ and Z₃ represents a nitrogen atom,
R₂ represents an aryl or heteroaryl group,
X represents a linear or branched alkylene chain having from 1 to 6 carbon atoms, wherein one or two of the carbon atoms may be replaced by an oxygen atom, a cycloalkylene group, an arylene group, a heteroarylene group or an SO₂ group,
R₁ represents a group of formula (II): 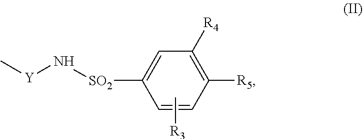
wherein:
Y represents a C═O or CH₂ group,
R₄ represents a hydrogen atom and R₅ represents hydrogen, —NR₆R′₆, or —CH₂—NR₆R′₆ wherein each of R₆ and R′₆, which may be the same or different, independently represents hydrogen or a linear or branched (C₁-C₆)alkyl group substituted by one or more aryl, heteroaryl, aryloxy, heteroaryloxy, arylthio, heteroarylthio, heterocycloalkyl or —NR₉R′₉ groups wherein:
R₉ and R′₉, which may be the same or different, are selected from hydrogen, linear or branched (C₁-C₆)alkyl, linear or branched (C₁-C₆)alkoxy, aryl and heteroaryl,
or R₉ and R′₉ form a saturated or unsaturated cyclic or bicyclic group optionally containing a hetero atom selected from oxygen, nitrogen and sulphur, wherein one or more of the ring members may represent a C═O group and wherein the cyclic or bicyclic group may be optionally substituted by 1 to 3 groups selected from linear or branched (C₁-C₆)alkyl optionally substituted by a hydroxy or amino group, linear or branched (C₁-C₆)alkoxy, hydroxy, carboxy, formyl, nitro, cyano, amino, linear or branched (C₁-C₆)polyhaloalkyl, alkyloxycarbonyl and halogen,
or R₄ and R₅, together with the two carbon atoms carrying them, form an aromatic or non-aromatic ring having 5 or 6 ring members, including a nitrogen atom in the position para to the SO₂ group, which in addition to the nitrogen atom may contain a further nitrogen atom and/or an SO₂ group, wherein the ring may be optionally substituted by an R₆ group as defined hereinbefore,
R₃ represents halogen, NO₂, hydrogen, a linear or branched (C₁-C₆)alkyl group substituted by one or more aryl, heteroaryl, aryloxy, heteroaryloxy, arylthio, heteroarylthio, heterocycloalkyl or —NR₉R′₉ groups, SO₂—R₈, a linear or branched (C₁-C₆)alkyl group, or a linear or branched (C₃-C₆)alkoxy group,
wherein R₈ represents an amino group or a linear or branched (C₁-C₆)alkyl group optionally substituted by one or more halogen atoms,
it being understood that:
“aryl” means a phenyl, naphthyl or biphenyl group,
“heteroaryl” means a mono- or bi-cyclic group having at least one aromatic moiety and having from 5 to 10 ring members, which may contain from 1 to 3 hetero atoms selected from oxygen, sulphur and nitrogen,
“heterocycloalkyl” means a mono- or bi-cyclic non-aromatic group having from 4 to 10 ring members, which may contain from 1 to 3 hetero atoms selected from oxygen, sulphur and nitrogen,
“cycloalkyl” means a mono- or bi-cyclic non-aromatic group containing from 4 to 10 ring members,
wherein the aryl, heteroaryl, heterocycloalkyl and cycloalkyl groups may be optionally substituted by from 1 to 3 groups selected from linear or branched (C₁-C₆)alkyl optionally substituted by a hydroxy or amino group, linear or branched (C₁-C₆)alkoxy, hydroxy, carboxy, formyl, nitro, cyano, amino, linear or branched (C₁-C₆)polyhaloalkyl, alkyloxycarbonyl and halogen, and
“arylene”, “heteroarylene” and “cycloalkylene” mean, respectively, a bivalent aryl, heteroaryl and cycloalkyl group as defined above,
and
its enantiomers, diastereoisomers, and addition salts thereof with a pharmaceutically acceptable acid or base.

19- The compound of claim 18, wherein Y represents a C═O group.

20- The compound of claim 18, wherein R₃ represents an NO₂ group.

21- The compound of claim 18, wherein X—R₂ represents a ([1,1′-biphenyl]-2-yl)methyl group optionally substituted by one or more halogen atoms.

22- The compound of claim 18, wherein R₄ represents a hydrogen atom.

23- The compound of claim 18, wherein R₆ represents 1-(N,N-dimethylamino)-4-(phenylsulphanyl)-butan-3-yl.

24- The compound of claim 18, wherein R′₆ represents a hydrogen atom.

25- The compound of claim 18 which is selected from:
N-({2-[(4′-chloro[1,1′-biphenyl]-2-yl)methyl]-1,2,3,4,10,10a-hexahydropyrido-[4′,3′:4,5]pyrrolo[1,2-a]pyrazin-8-yl}carbonyl)-4-({(1R)-3-(dimethylamino)-1-[(phenylsulphanyl)methyl]propyl}amino)-3-nitrobenzenesulphonamide,
N-({8-[(4′-chloro[1,1′-biphenyl]-2-yl)methyl]-6,7,8,9,9a,10-hexahydropyrido-[2′,3′:4,5]pyrrolo[1,2-a]pyrazin-2-yl}carbonyl)-4-({(1R)-3-(dimethylamino)-1-[(phenylsulphanyl)methyl]propyl}amino)-3-nitrobenzenesulphonamide,
N-({7-[(4′-chloro[1,1′-biphenyl]-2-yl)methyl]-5,5a,6,7,8,9-hexahydropyrido-[3′,2′:4,5]pyrrolo[1,2-a]pyrazin-3-yl}carbonyl)-4-({(1R)-3-(dimethylamino)-1-[(phenylsulphanyl)methyl]propyl}amino)-3-nitrobenzenesulphonamide,
N-({3-[(4′-chloro[1,1′-biphenyl]-2-yl)methyl]-2,3,4,4a,5,6-hexahydro-1H-pyrazino[1,2-a][1,5]naphthyridin-8-yl}carbonyl)-4-({(1R)-3-(dimethylamino)-1-[(phenylsulphanyl)methyl]propyl}amino)-3-nitrobenzenesulphonamide,
N-({8-[(4′-chloro[1,1′-biphenyl]-2-yl)methyl]-6,6a,7,8,9,10-hexahydro-5H-pyrazino[1,2-a][1,7]naphthyridin-3-yl}carbonyl)-4-({(1R)-3-(dimethylamino)-1-[(phenylsulphanyl)methyl]propyl}amino)-3-nitrobenzenesulphonamide,
N-({8-[(4′-chloro[1,1′-biphenyl]-2-yl)methyl]-6,6a,7,8,9,10-hexahydro-5H-pyrazino[1,2-a][1,8]naphthyridin-3-yl}carbonyl)-4-({(1R)-3-(dimethylamino)-1-[(phenylsulphanyl)methyl]propyl}amino)-3-nitrobenzenesulphonamide,
and addition salts thereof with a pharmaceutically acceptable acid or base.

26- A pharmaceutical composition comprising as active ingredient a compound of claim 18, or an addition salt thereof with a pharmaceutically acceptable acid or base, in combination with one or more pharmaceutically acceptable excipients.

27- A method of treating a condition involving a defect in apoptosis in a subject in need thereof, comprising administration of an effective amount of a compound of claim 18.

28- A method of treating cancer in a subject in need thereof, comprising administration of an effective amount of a compound of claim 18.

29- The method of claim 28, wherein the cancer is selected from cancers of the bladder, brain, breast and uterus, chronic lymphoid leukaemias, cancers of the colon, oesophagus and liver, lymphoblastic leukaemias, follicular lymphomas, melanomas, malignant haemopathies, myelomas, ovarian cancers, non-small-cell lung cancers, prostate cancers and small-cell lung cancers.

30- A composition comprising a compound of claim 18 and an anti-cancer agent selected from genotoxic agents, mitotic poisons, anti-metabolites, proteasome inhibitors and kinase inhibitors.

31- A method of treating cancer in a subject in need thereof, comprising administration of an effective amount of a composition of claim 30.

32- A method of treating cancer in a subject in need thereof, comprising administration of an effective amount of a compound of claim 18 in combination with radiotherapy.
